(19) United States
(12) Patent Application Publication
(10) Pub. No.: US 2023/0258080 A1
(43) Pub. Date: Aug. 17, 2023

(54) RESERVOIR FLUID PROPERTY ESTIMATION USING MUD-GAS DATA

(71) Applicant: Equinor Energy AS, Stavanger (NO)

(72) Inventors: Tao YANG, Stavanger (NO); Margarete Maria KOPAL, Stavanger (NO); Ibnu Hafidz ARIEF, Stavanger (NO); Gulnar YERKINKYZY, Stavanger (NO); Knut ULEBERG, Stavanger (NO)

(73) Assignee: Equinor Energy AS, Stavanger (NO)

(21) Appl. No.: 18/014,575

(22) PCT Filed: Jul. 2, 2021
(86) PCT No.: PCT/NO2021/050158

§ 371 Date: Jan. 5, 2023

(30) Foreign Application Priority Data
Jul. 6, 2020 (GB) 2010337.0

(51) Int. Cl. E21B 49/08; G01N 33/28; G01N 30/02

(52) U.S. Cl. CPC E21B 49/087; G01N 33/2823; G01N 30/02; G01N 2030/025

(57) ABSTRACT

A method is disclosed for generating a machine learning model to predict a reservoir fluid property, such as gas-oil ratio or density, based on standard mud-gas and petrophysical data. It has been found that this model predicts these reservoir fluid properties with an accuracy that is close to that which can be achieved using advanced mud-gas data. This is advantageous, as than standard mud-gas data and petrophysical data is much more readily available than advanced mud-gas data.

The present disclosure relates to a logging technique for use whilst drilling a borehole, and particularly to a technique utilising mud-gas data to predict reservoir fluid properties.

Drilling fluid is a fluid used to aid the drilling of boreholes into the earth. The main functions of drilling fluid include providing hydrostatic pressure to prevent formation fluids from entering into the well bore, keeping the drill bit cool and clean during drilling, carrying out drill cuttings, and suspending the drill cuttings while drilling is paused and when the drilling assembly is brought in and out of the hole.

Drilling fluids are broadly categorised into water-based drilling fluid, non-aqueous drilling fluid, often referred to as oil-based drilling fluid, and gaseous drilling fluid. Liquid drilling fluids, i.e. water-based drilling fluid or non-aqueous drilling fluid, are commonly referred to as “drilling mud”.

Mud-gas logging entails gathering data from hydrocarbon gas detectors that record the levels of gases brought up to the surface in the drilling mud during a bore drilling operation.

Conventionally, mud-gas logging is used to identify the location of oil and gas zones as they are penetrated, which can be identified by the presence of hydrocarbon gas in the mud system. This may be used to provide a general indication of the type of reservoir, as well as to determine where to take downhole fluid samples for more detailed analysis of the fluid composition. The presence of hydrocarbon gas may be detected, for example, with a total gas detector.

Once the presence of hydrocarbon gas is detected, its composition may be examined for example with a gas chromatograph.

The most common gas component present is usually methane (C₁). The presence of heavier hydrocarbons, such as C₂ (ethane), C₃ (propane), C₄ (butane) and C₅ (pentane) may indicate an oil or a “wet” gas zone. Heavier molecules, up to about C₇ (heptane), may also be detectable, but are typically present only in very low concentrations. Consequently, the concentrations of these hydrocarbons are often not recorded.

There are two types of mud-gas data that can be collected, which are sometimes referred to a “standard” mud-gas logging, and “advanced” mud-gas logging. The equipment for standard mud gas logging and advanced mud gas logging are different.

For a standard mud gas system, the degasser does not usually have heating or uses constant volume gas separation. There is also only one sampling point of mud sample (“out”) and therefore it is not suitable for recycling correction. The measured gas composition is usually referred standard mud-gas data, which is not directly comparable to the actual C₁ to C₅ composition of the reservoir fluid sample.

For an advanced mud gas system, the degasser has heating and usually uses a constant volume for gas separation. There are two sampling points of mud samples (“out” and “in”), and therefore it is possible to perform recycling correction. The measured gas composition is usually referred advanced mud-gas data.

When generating advanced mud-gas data, in order to make the data closely correspond to the actual reservoir fluid C₁ to C₅ concentrations, two correction processes are applied to the “raw” mud-gas data from the advanced mud gas logging system.

Firstly, a recycling correction is made to eliminate contamination of the gas by gases originating from previous injections of the drilling mud. This correction is applied based on a separate mud-gas measurement that was taken before the drilling mud was injected into the drilling string.

Secondly, an extraction efficiency correction step is applied to increase the concentration of intermediate components (from C₂ to C₅), such that the concentration of these components, relative to the C₁ concentration, more closely resemble the relative compositions of a corresponding reservoir fluid sample. The extraction efficiency correction is applied based on the type of drilling mud used for the borehole.

In the past, the advanced mud gas data would have been examined to estimate certain fluid properties of the reservoir fluid using broad, empirical correlations between the advanced mud-gas composition and certain fluid properties of the reservoir fluid. For example, extremely dry gas reservoirs should comprise mostly C₁ and not much C₂₊, e.g. with each of the C₁/C₂, C₁/C₃, C₁/C₄ and C₁/C₅ ratios (for the raw mud-gas data) being greater than 50. Wet gas reservoirs will often have ratios between 20 and 50, and oil reservoirs will have ratios between 2 and 20.

Recently, an advanced machine learning model has been developed, making it possible to predict reservoir fluid properties much more accurately from the advanced mud-gas data, even where those properties are dependent upon the oil part (C₇₊) of the fluid which is not measured by the mud-gas data.

Details of how such a machine learning model was trained to determine a gas-oil ratio of the reservoir fluid based on the advanced mud-gas data can be found in the paper Tao Yang et. al. (2019), “A Machine Learning Approach to Predict Gas Oil Ratio Based on Advanced Mud Gas Data”. Society of Petroleum Engineers. doi:10.2118/195459-MS

Advantageously, this model can be used to generate a substantially continuous log of the respective reservoir fluid property. This was not previously possible, and in the past, it was necessary to rely on downhole fluid samples. Furthermore, the model allows reservoir fluid property predictions to be made at a very early stage of the drilling process and without needing to interrupt the drilling process, as might be required to take downhole fluid samples or the like.

This model has been found to be very useful, but is limited in that it requires the availability of advanced mud-gas data. A need exists for a technique that can be used when advanced mud-gas data is not available.

The present invention provides a method of generating a model for predicting at least one property of a fluid at a sample location within a hydrocarbon reservoir, comprising:

providing a training data set comprising input data and target data, the input data comprising mud-gas data and petrophysical data for each of a plurality of sample locations, and the target data comprising the at least one property of the fluid for each of the plurality of sample locations; and

generating a model using the training data set such that the model can be used to predict the at least one property of the fluid at the sample location based on measured mud-gas data and measured petrophysical data for the sample location,

wherein a drilling fluid recycling correction has not been applied to the mud-gas data.

It is a commonly held belief within the oil and gas industry that petrophysical data provides only a qualitative indication of a reservoir fluid. The data usually predicts lean gas with good certainty but has reduced accuracy when used to distinguish rich gas condensate and oil. However, it has been identified that, by supplementing standard mud-gas data with petrophysical data, it is possible to provide an estimation of certain reservoir fluid properties with an accuracy that is close to the accuracy that can be achieved using advanced mud-gas data alone.

This is particularly advantageous where it is desirable to generate fluid property logs for large amounts of existing and new wells because standard mud-gas data and petrophysical data are collected for almost all the wells, including both exploration and production wells. Whereas, the additional cost of collecting advanced mud-gas data, and particularly of having the two sets of mud-gas analysis tools required to perform the recycling correction, means that it is often only collected when drilling some exploration wells. The number of wells with advanced mud gas data only represents a small portion of the total wells with standard mud-gas data and petrophysical data.

Additionally, the above technique allows for reservoir fluid property logs to be generated for new wells at a reduced cost, as it does not require the additional costs associated with collecting advanced mud-gas data. Importantly in this regard is that petrophysical data can be collected as a substantially continuous log, similar to mud-gas data. This contrasts with downhole fluid sample data, which requires interruption of the drilling process, adding significant additional costs to the drilling process.

In some embodiments, the input data may not comprise downhole fluid sampling data, and the model may not require downhole fluid sampling data as an input to predict the at least one property of the fluid at the sample location.

The method is preferably a computer-implemented method, and generating the model may comprise instructing a machine learning algorithm to generate the model using the training data set such that the model can be used to predict the at least one property of the fluid at the sample location based on measured mud-gas data for the sample location.

The at least one property is preferably a property influenced by the oil-related components of the fluid. That is to say, a property that not solely the product of the gaseous hydrocarbons within the fluid, whose composition can be predicted based on the mud-gas data.

The at least one property may comprise a density of the fluid at the sample location. It will be appreciated that the density may be calculated either at atmospheric conditions or reservoir conditions (e.g. taking into account the oil formation volume factor).

The at least one property may comprise a gas-oil ratio. That is to say, a ratio between the quantity of gaseous hydrocarbon and the quantity of liquid hydrocarbon, which is normally determined at surface conditions. The gas-oil ratio is preferably a volume ratio. The gas-oil ratio may be a single-flash gas-oil measurement. However, any suitable gas-oil measurement may be used.

The at least one property may comprise a saturation pressure of the fluid at the sample location. That is to say, the pressure at which a secondary phase will appear with pressure depletion.

The at least one property comprises a formation volume factor of the fluid at the sample location. That is to say, the ratio of the volume of the fluid at reservoir (in-situ) conditions to the volume of the fluid at surface conditions.

The at least one property may comprise a concentration of a hydrocarbon within the fluid at the sample location. The hydrocarbon may be a hydrocarbon that is not included within the mud-gas data. For example, the hydrocarbon may be a C₇₊ hydrocarbon. That is to say, the hydrocarbon may be a C₇ hydrocarbon or may be a hydrocarbon heavier that C₇, e.g. a C₈ or heavier hydrocarbon. The hydrocarbon may be a hydrocarbon that is substantially an oil at reservoir conditions. The concentration of the hydrocarbon may be an absolute concentration (e.g. a molar concentration), or may be a relative concentration (e.g. a ratio compared to C₁), or may be an otherwise normalised concentration.

The reservoir may be a gas reservoir, a multiphase reservoir or an oil reservoir.

The at least one property for each sample location may be determined from reservoir fluid properties data associated with the sample location. The reservoir fluid properties data may comprise measured composition data for a fluid at the sample location. The reservoir fluid properties data may contain the composition of C₁ to C₇₊ hydrocarbons at the sample location, and preferably C₁ to C₂₀₊ hydrocarbons, and more preferably C₁ to C₃₆₊ hydrocarbons at the sample location. As used herein, the “Cₓ₊” notation should be understood as meaning Cₓ or heavier hydrocarbons.

The mud-gas data of the training data set may comprise measured mud-gas data for the sample location, i.e. measured standard mud-gas data for the sample location.

The measured mud-gas data may be indicative of a composition of gases released from drilling fluid used whilst drilling through the sample location (i.e. passing through a drill bit performing the drilling). The measured mud-gas data may be indicative of a concentration of at least C₁ to C₄ gaseous hydrocarbons, and preferably at least C₁ to C₅ gaseous hydrocarbons, that was released from drilling mud.

As discussed above, the mud-gas data preferably does not comprise advanced mud-gas data, i.e. where the mud-gas data has not been corrected so as to correspond to the gaseous hydrocarbon composition of the fluid at the sample location.

A drilling fluid recycling correction refers to correcting the mud-gas data to remove errors due to gases released from previous drilling operations, such as due to recycling of the drilling fluid. Typically, this would require a reference mud-gas data measurement collected before injection of the drilling mud into the drilling string.

Optionally, an extraction efficiency correction has not been applied to the mud-gas data.

An extraction efficiency correction refers to correcting the mud-gas data (C₁ to C₅) to closely correspond to reservoir fluid composition due to different hydrocarbons components have different abilities to vaporize from the drilling mud.

Where an extraction efficiency correction has not been applied, the training data may additionally comprise drilling mud compositional data. This may allow the machine learning model to correct the data within the model generated by the machine learning algorithm.

Alternatively, an extraction efficiency correction may have been applied to the standard mud-gas data. Often, the type of drilling mud used for a well is known and in many cases the extraction efficiency corrections can be estimated by Equation of State (EOS) simulation or approximated by testing. Consequently, even when using standard mud-gas data, it may be possible to retrospectively apply an extraction efficiency correction to standard mud-gas data.

Optionally, the mud-gas data was collected without the use of heating. Whilst standard mud-gas data may use heating; heating has often not been used when collecting mud-gas data. Consequently, where it is desirable to utilise the model to examine existing wells, a model trained using mud-gas data collected without the use of heating is particularly useful.

The petrophysical data may comprise any one or more of: bulk density, neutron porosity, resistivity data, acoustic data, natural gamma ray, nuclear magnetic resonance data, as well as slowing down time and gamma ray spectroscopy data from pulsed neutron measurements, and the like. Optionally, the petrophysical data may comprise two or more of these data types.

Generating the model may comprise: training a machine learning algorithm with a first subset of the training data set; and testing the machine learning algorithm with a second, disjoint subset of the training data set. The first subset preferably comprises at least 50% of the samples of the training data set. The second subset preferably comprises at least 10% of the samples of the training set.

Viewed from a second aspect, the present invention provides a computer-based model for predicting at least one property of a fluid at a sample location within a hydrocarbon reservoir based on measured mud-gas data and measured petrophysical data for that sample location, the computer-based model having been generated by the method above.

Viewed from third aspect, the present invention provides a tangible computer-readable medium storing the computer-based model.

Viewed from a fourth aspect, the present invention provides a method of predicting a value of a property of a fluid at a sample location within a hydrocarbon reservoir, the method comprising: receiving measured mud-gas data and measured petrophysical data for the sample location; and predicting the value of the property of the fluid at the sample location by supplying the measured mud-gas data and the measured petrophysical data to the computer-based model.

The method may further comprise determining a quality for the measured mud-gas data and/or the measured petrophysical data.

The method may further comprise generating an indication of confidence associated with the predicted value of the fluid property. The indication of confidence may be a numerical indication, but other indications may be used, such as colour indications (e.g. red/yellow/green), or word indications (e.g. “good”/“poor”).

The indication of confidence may be based on the quality of the measured mud-gas data and/or the measured petrophysical data.

For a single data point, the indication of confidence may be reduced by one or more of a C₁, C₄ or C₅ concentration that is below a respective predetermined threshold.

Where standard mud-gas data is taken at a series of locations at different depths, the indication of confidence may be reduced by fluctuations of a component concentration of the mud-gas data greater than a threshold amplitude within a predetermined depth range.

Where standard mud-gas data and/or the petrophysical data is taken at a series of locations at different depths, the indication of confidence may be reduced by the missing of a predetermined number of preceding measurements or over a predetermined depth range.

Viewed from a fifth aspect, the present invention provides a method of predicting a value of a fluid property of a fluid along a length of a well through a hydrocarbon reservoir, the method comprising: predicting a value of a fluid property of a fluid at a plurality of sample locations along a length of a well using the method above.

The method may comprise: displaying, using an electronic display screen, a graph plotting the predicting value of the fluid property against a location of the respective sample location for each of the plurality of sample locations along the length of the well.

The method may further comprise: indicating, using the electronic display screen, an indication of confidence associated with one or more of the predicted value. For example, the indication of confidence may be illustrated numerically, verbally, chromatically or iconographically.

All of the method described above, i.e. the methods of the first, fourth and fifth aspects may be performed in any suitable and desired way and on any suitable and desired platform. In a preferred embodiment the methods are each a computer-implemented method, e.g. the steps of the method are performed by processing circuitry.

The methods in accordance with the present invention may be implemented at least partially using software, e.g. computer programs. It will thus be seen that when viewed from further aspects the present invention provides computer software specifically adapted to carry out the methods described herein when installed on a data processor, a computer program element comprising computer software code portions for performing the methods described herein when the program element is run on a data processor, and a computer program comprising code adapted to perform all the steps of a method or of the methods described herein when the program is run on a data processing system.

The present invention also extends to a computer software carrier comprising such software arranged to carry out the steps of the methods of the present invention. Such a computer software carrier could be a physical storage medium such as a ROM chip, CD ROM, DVD, RAM, flash memory or disk, or could be a signal such as an electronic signal over wires, an optical signal or a radio signal such as to a satellite or the like.

It will further be appreciated that not all steps of the methods of the present invention need be carried out by computer software and thus from a further broad embodiment the present invention provides computer software and such software installed on a computer software carrier for carrying out at least one of the steps of the methods set out herein.

The present invention may accordingly suitably be embodied as a computer program product for use with a computer system. Such an implementation may comprise a series of computer readable instructions, which may be fixed on a tangible, non-transitory medium, such as a computer readable medium, for example, diskette, CD ROM, DVD, ROM, RAM, flash memory or hard disk. It could also comprise a series of computer readable instructions transmittable to a computer system, via a modem or other interface device, over either a tangible medium, including but not limited to optical or analogue communications lines, or intangibly using wireless techniques, including but not limited to microwave, infrared or other transmission techniques. The series of computer readable instructions embodies all or part of the functionality previously described herein.

Those skilled in the art will appreciate that such computer readable instructions can be written in a number of programming languages for use with many computer architectures or operating systems. Further, such instructions may be stored using any memory technology, present or future, including but not limited to, semiconductor, magnetic or optical, or transmitted using any communications technology, present or future, including but not limited to optical, infrared or microwave. It is contemplated that such a computer program product may be distributed as a removable medium with accompanying printed or electronic documentation, for example, shrink wrapped software, pre-loaded with a computer system, for example, on a system ROM or fixed disk, or distributed from a server or electronic bulletin board over a network, for example, the Internet or World Wide Web.

Certain preferred embodiments of the present disclosure will now be described in greater detail, by way of example only and with reference to the accompanying drawings, in which:

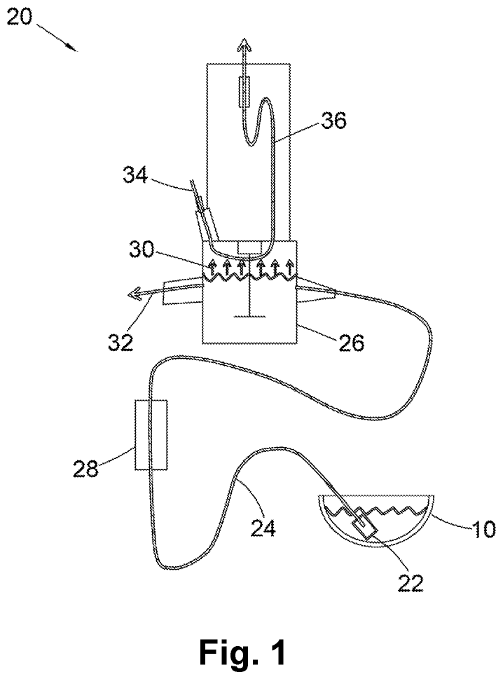

FIG. 1 is a schematic illustration of a mud-gas analysis tool; and

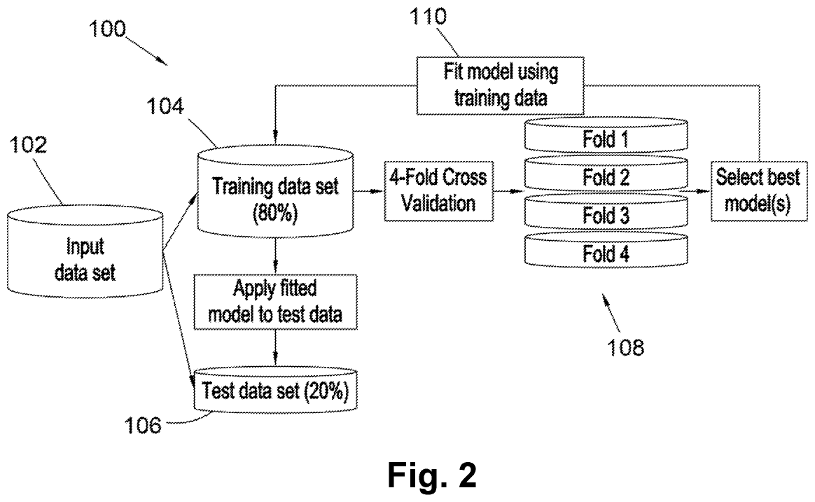

FIG. 2 illustrates a workflow for a machine learning algorithm to generate a first model for predicting a gas oil ratio using a training data set;

An exemplary standard mud-gas analysis tool 20 is shown schematically in FIG. 1.

The tool 20 is coupled to a flow line 10 containing drilling mud returned from a borehole of a well. As discussed above, the drilling mud may be water-based mud or oil-based mud.

The tool 20 comprises a sampling probe 22 disposed with respect to the flow line 10 so as to collect a sample 24 of the drilling mud from the flow line 10. The drilling mud sample 24 is preferably a continuous sample, i.e. such that a portion of the flow of drilling mud within the flow line 10 is diverted through the mud-gas analysis tool 20.

The drilling mud sample 24 is supplied to a gas-separation chamber 26 where at least a portion of the gas carried by the drilling mud is released. The sample of drilling mud may optionally be heated by a heater 28 upstream of the gas-separation chamber 26. Heating the drilling mud sample 24 helps to release the gas from the drilling mud sample 24. Typically, the mud sample 24 is not heated and the temperature are typically ranging from 10° C. to 60° C. However, in some implementations, heating is used to 80° C. to 90° C.

The released gas 30 is directed from the separation chamber 26 to a gas analysis unit (not shown), while the degassed mud 32 is returned to the flow line 10 or to another location for re-use.

The gas analyser may comprise a total gas detector, which may provide a basic quantitative indication as to how much gas is being extracted from the drilling mud by the tool 20. Total gas detection typically incorporates either a catalytic filament detector, also called a hotwire detector, or a hydrogen flame ionization detector.

A catalytic filament detector operates on the principle of catalytic combustion of hydrocarbons in the presence of a heated platinum wire at gas concentration below the lower explosive limit. The increasing heat due to combustion causes a corresponding increase in the resistance of the platinum wire filament. This resistance increase may be measured through the use of a Wheatstone bridge or equivalent detection circuit.

A hydrogen flame ionization detector functions on the principle of hydrocarbon molecule ionization in the presence of a very hot hydrogen flame. These ions are subjected to a strong electrical field resulting in a measurable current flow.

The gas analysis device may additionally or alternatively comprise an apparatus for detailed analysis of the hydrocarbon mixture. This analysis is usually performed by a gas chromatograph. However, several other detecting devices may also be utilised including a mass spectrometer, an infrared analyser or a thermal conductivity analyser.

A gas chromatograph is a rapid sampling, batch processing instrument that provides a proportional analysis of a series of hydrocarbons. Gas chromatographs can be configured to separate almost any suite of gases, but typically oilfield chromatographs are designed to separate the paraffin series of hydrocarbons from methane (C₁) through pentane (C₅) at room temperature, using air as a carrier. The chromatograph will report (in units or in mole percent) the quantity of each component of the gas detected.

A carrier gas stream 34, commonly comprising air, may be supplied to the separation chamber 26 and mixed with the released gas 30 to form a gas mixture 36 that is supplied to the gas analysis unit. The carrier gas stream 34 provides a continuous flow of carrier gas in order to provide a substantially continuous flow rate of the gas mixture 36 from separation chamber 26 to the gas analysis unit. Additionally, in the case of a gas analyser comprising a combustor, the use of air as the carrier gas may provide the necessary oxygen for combustion.

In some arrangements, the tool 20 may be configured to detect and/or remove H₂S from the gas to prevent adverse effects that could influence hydrocarbon detection.

In some embodiments, non-combustibles gases, such as helium, carbon dioxide and nitrogen, can be detected by the gas analyser in conjunction with the logging of hydrocarbons.

The following technique seeks to utilise a machine learning algorithm to produce a model that accurately estimates certain properties relating to the reservoir fluid, in particular the gas-oil ratio and the density of the reservoir fluid, based on the standard mud-gas data and other petrophysical data.

FIG. 2 illustrates a workflow 100 for training the machine learning algorithm in order to generate a model for prediction of a gas-oil ratio of a reservoir based on measured standard mud-gas data.

In the following example, an input data set 102 is used as a training data set and comprises data relating to a plurality of reservoir samples.

The input data set comprises reservoir fluid properties data from a large number of reservoir fluid samples. Reservoir samples may be obtained, for example, by downhole fluid sampling. However, other techniques could also be used, for example by taking a sample of well fluid after the well has been completed.

The reservoir fluid properties data should include at least hydrocarbon composition data, which may be either in the form of direct measurements of the concentration of each hydrocarbon component within the sample, typically covering C₁ to C₃₆₊ hydrocarbons. In some embodiments, the concentration data may be in the form of relative data (e.g. as a ratio of compositions of different hydrocarbons) or may be otherwise normalised. The reservoir fluid properties data may optionally also include concentrations of one or more other constituents within the well.

The reservoir fluid properties data may include one or more derived properties of the reservoir fluid sample. Such derived properties may include the target property to be determined by the machine-learning algorithm, e.g. a gas-oil ratio in this case. Other derived properties may include a density of the fluid.

The reservoir fluid properties data is sometimes referred to as PVT data, as it is commonly obtained in a pressure-volume-temperature (PVT) laboratory, where researchers will employ various instruments to determine reservoir fluid behaviour and properties from the reservoir samples.

The input data set 102 further comprises measured standard mud-gas data for each PVT sample at the same reservoir depth. The measured standard mud-gas data comprises measured hydrocarbon composition data for gas released from the drilling fluid from the sample location.

It will be appreciated that there is a lag-time between the drill bit passing through the sample location, and when the mud reaches the surface and is analysed. However, workers in this field will be familiar with the procedures for calculating the lag time to determine the depth to which the mud-gas sample corresponds. Therefore, this will not be discussed in detail.

The composition data for the mud-gas preferably comprises data for at least C₁ to C₄ hydrocarbons, and preferably at least C₁ to C₅ hydrocarbons (as is the case in the present example). In some cases, concentrations for up to C₇ or greater hydrocarbons may be included.

The composition data may be stored either as a direct measurement of concentration (e.g. measured in ppm or similar units), or alternatively as a relative concentration (e.g. as a proportion of another hydrocarbon, usually C₁). In some 10 embodiments, the composition data may be normalised.

The measured standard mud-gas data is “raw” mud-gas data, i.e. it has not been corrected for recycling or extraction efficiency. This is important as the use of “raw” mud-gas data will allow the subsequent model to be utilised more widely, where advanced mud-gas data is not available.

The input data set 102 further comprises measured petrophysical data for each PVT sample at the same reservoir depth. The petrophysical data may comprise any one or more of: bulk density, neutron porosity, resistivity data, acoustic data, natural gamma ray, nuclear magnetic resonance data, as well as slowing down time and gamma ray spectroscopy data from pulsed neutron measurements, and the like.

The input data set 102 comprises target data and input data for each sample that passed the screening. The target data corresponds to the desired output of the model. The input data corresponds to the data that will be input into the eventual model.

The target data in this example is a gas-oil ratio, and in this example is the single-flash gas-oil measurement of the sample. As discussed above, this data is stored as part of the reservoir properties data within the initial data set. Alternatively, other measurements of gas-oil ratio may be used, or a gas-oil ratio may be derived from the reservoir composition data, i.e. based on the concentrations of the various hydrocarbons.

The input data is standard mud-gas data, i.e. data indicative of the composition of gases released from the drilling fluid from the sample location, and at least one type of petrophysical data, e.g. bulk density, and neutron porosity.

As mentioned above, the measured mud-gas data comprises “raw” mud-gas data, i.e. it has not been corrected for recycling or extraction efficiency.

Whilst it is not possible to apply a recycling correction after collection of the data, nor is it possible to account for the lack of heating (if heating was not used), it may be possible to apply a retrospective extraction efficiency correction. This is because the composition of the drilling mud for a particular well is usually known, and the extraction efficiency correction factors for that particular drilling fluid can be estimated either from EOS simulation or may be approximated by experiment. Results show that the temperature dependent extraction efficiency correction far outweighs recycling corrections.

Consequently, the mud-gas data used for the input data set 102 preferably comprises standard mud-gas data where an extraction efficiency correction has been applied.

Next, a model generation is performed, in which a model is generated and validated based on the input data set 102.

The input data set 102 is first divided into a training data set 104 and a testing data set 106. The input data set 102 is preferably curated such that at least the testing data set 106 contains data that spans the various classes of the input data set 102 as a whole (e.g. dry gas reservoirs, wet gas reservoirs, oil reservoirs).

Typically, at least 50% of the input data set 102 should be used for training, and at least 10% of the input data set 102 should be used for testing. Common ratios include 50:50, 70:30, 75:25, 80:20, 90:10. However, it will be appreciated that other divisions may be used instead.

Generally the larger the training data set, the more accurate the model will be. However, if too small a test data set is used (or indeed if no test data set is used) then it is not possible to confidently verify the accuracy of the model, e.g. making it difficult to detect an over-fitted model (only accurate for the specific training data).

To generate a model, a machine learning algorithm is provided with the training data set 104, and a set of training parameters to control the machine learning algorithm.

In one example, Gaussian Process Regression and Random Forest were found to be best performing models. However, it will be appreciated that any suitable algorithm may be used, such as Universal Kriging, KMean or Elastic Net algorithms. Those operating within this field will be familiar with the procedures for selecting and utilising a machine learning algorithm. Therefore, this will not be discussed in detail.

Model validation 108, e.g. cross-validation, may then then be performed. During the model validation 108, the model is tested to determine how well it predicts new data that was not used in estimating the model, in order to flag problems such as over fitting or selection bias. Model validation 108 is an optional step.

Cross-validation involves partitioning the training data set 104 into complementary subsets, performing the model fitting using one subset of the training data set 104, and validating the analysis on the other subset of the training data set 104. To reduce variability, most methods use multiple rounds of cross-validation, performed using different partitions, and the validation results are combined (e.g. averaged) over the rounds to give an estimate of the model's predictive performance (e.g. a mean average prediction error, MAPE).

In this example K-fold cross-validation, and particularly 4-fold cross-validation is used. In K-fold cross-validation, the training data 104 is separated in K disjoint subsets (in this case, four), known as “folds”. Then, cross-validation is performed by training the model on all of the data except for one fold, and validating the trained model using the fold that was not used for training. The best model is then selected as the model having the best predictive performance, e.g. the lowest MAPE.

A first testing step 110 is then performed, in which the model is tested using the training data set 104 as a whole.

A second testing step 112 is then performed, in which the model is tested using the test data set 106. As discussed previously, this is a curated set of data that is broadly representative of the data as a whole, and was not used during the generation of the model.

The model has been found to predict a gas-oil ratio of the reservoir fluid based on C₁ to C₅ standard mud-gas data and the petrophysical data with MAPE that is close to that achieved using a model based on C₁ to C₅ advanced mud-gas data.

Understanding the quality of the measured mud-gas data is important before performing a fluid property (e.g. gas-oil ratio) prediction because the mud-gas data quality will significantly impact prediction accuracy. The following characteristics of the mud-gas data values have been identified as indicating low quality or unreliable data:
Large fluctuations of a component within a small depth range.
First observations after missing measurements.
C₁ content below a given threshold.
C₄ or C₅ content below a given threshold.

To quantify the quality of the mud-gas data, the inventors derived a quality control metric (QC metric) which ranged from 0 to 1. High quality mud-gas data would have QC metric value close to 1. If one or more of the above factors are found, then the QC metric would be reduced. Low-quality mud-gas data was indicated by QC metric close to 0. A single numeric quality measure between 0 and 1 can be plotted side-by-side with a predicted fluid property log (as will be discussed below) to visualize the confidence level associated with each prediction, based on mud-gas data quality.

Samples having a higher QC metric correspond closely, whilst samples having a lower QC metric have poor correspondence. Thus, these factors provide a useful indication of the accuracy of a prediction of the gas-oil ratio.

Mud-gas data and petrophysical data are both generated continuously during the drilling process. Therefore, by applying the machine learning model to the mud-gas data and petrophysical data, it is possible to provide, at an early stage of the well installation procedure, a continuous log for the well bore of the predicted reservoir property, e.g. gas-oil ratio or fluid density. This is something that has not been possible previously until much later in the process.

Whilst the above examples have been described in the context of a gas-oil ratio as the target reservoir fluid property, the same technique may also be employed to create a model for estimating other reservoir fluid properties of the reservoir fluid at a sample location, based on measured mud-gas data. Exemplary reservoir fluid properties include a fluid density of the reservoir fluid, either a stock tank oil density or a live reservoir density, a saturation pressure of the reservoir fluid, and a formation volume factor of the reservoir fluid.

Furthermore, a similar technique may be used to train a model to estimate the reservoir fluid composition and corresponding C₇₊ fraction properties. This is advantageous, as this information can be used to for an equations of state (EOS) model calculation. The EOS model for a particular fluid is an expression that describes the relationship between pressure, temperature and volume of the fluid and can be used to predict the phase behaviour of the fluid in order to derive further properties thereof.

It is normally considered necessary to know at least the following properties of the fluid in order to determine the equations of state:
1) The absolute composition of each of the C₁ to C₆ hydrocarbons and the absolute composition of the C₇₊ hydrocarbons combined;
2) The average hydrocarbon density of the C₇₊ hydrocarbons; and
3) The average hydrocarbon molecular weight of the C₇₊ hydrocarbons.

When determining the equations of state for a fluid, the C₇₊ hydrocarbons are usually grouped together because these hydrocarbons usually remain in the liquid/oil phase. A standard C₇₊ characterisation method can split the C₇₊ into multiple pseudo components for EOS calculation.

Although individual fluid property models (like density and GOR) were developed in the first examples, it will be appreciated that a physical model could be generated that would calculate all fluid properties. The EOS model approach in the second example demonstrates a good solution for predicting all reservoir fluid properties.

Whilst preferred embodiments have been described above, it will be appreciated that these have been provided by way of example only, and the scope of the invention is to be limited only by the following claims.

1. A method of generating a model for predicting at least one property of a fluid at a sample location within a hydrocarbon reservoir, comprising:
providing a training data set comprising input data and target data, the input data comprising mud-gas data and petrophysical data for each of a plurality of sample locations, and the target data comprising the at least one property of the fluid for each of the plurality of sample locations; and
generating a model using the training data set such that the model can be used to predict the at least one property of the fluid at the sample location based on measured mud-gas data and measured petrophysical data for the sample location,
wherein a drilling fluid recycling correction has not been applied to the mud-gas data.

2. The method according to claim 1, wherein generating the model comprises instructing a machine learning algorithm to generate the model using the training data set.

3. The method according to claim 1, wherein the at least one property comprises a property influenced by the oil-related components of the fluid.

4. The method according to claim 1, wherein the at least one property comprises one or more of:
a density of the fluid at the sample location;
a gas-oil ratio of the fluid at the sample location;
a saturation pressure of the fluid at the sample location;
a formation volume factor of the fluid at the sample location; and
a concentration of C₇₊ hydrocarbons within the fluid at the sample location.

5. The method according to claim 1, wherein the mud-gas data of the training data set comprises measured standard mud-gas data for the sample location.

6. The method according to claim 5, wherein an extraction efficiency correction has been applied to the mud-gas data of the training data set.

7. The method according to claim 5, wherein an extraction efficiency correction has not been applied to the mud-gas data of the training data set, and wherein the training data comprise drilling mud compositional data.

8. The method according to claim 5, wherein the measured mud-gas data was collected without the use of heating.

9. The method according to claim 1, wherein the petrophysical data comprise one or more of:
bulk density;
neutron porosity;
resistivity data;
acoustic data;
natural gamma ray;
nuclear magnetic resonance data; and
gamma ray spectroscopy data.

10. A computer-based model for predicting at least one property of a fluid at a sample location within a hydrocarbon reservoir based on measured mud-gas data and measured petrophysical data for that sample location, the computer-based model having been generated by the method according to claim 1.

11. A tangible computer-readable medium storing the computer-based model according to claim 10.

12. A method of predicting a value of a property of a fluid at a sample location within a hydrocarbon reservoir, the method comprising:
receiving measured mud-gas data and measured petrophysical data for the sample location; and
predicting the value of the property of the fluid at the sample location by supplying the measured mud-gas data and the measured petrophysical data to the computer-based model according to claim 10.

13. A method of predicting a value of a fluid property of a fluid along a length of a well through a hydrocarbon reservoir, the method comprising:
predicting a value of a fluid property of a fluid at a plurality of sample locations along a length of a well using the method according to claim 12 for each sample location.

14. The method according to claim 13, further comprising:
displaying, using an electronic display screen, a graph plotting the predicting values of the fluid property against a location of the respective sample location for each of the plurality of sample locations along the length of the well.
